SEQUENCE LISTING (4 sequences)

SEQ ID NO: 1 - DNA, 36 bp, Artificial Sequence
  Primer
gagtgagtgcggccgcggtggtcgtcgtgcatccgt

SEQ ID NO: 2 - DNA, 34 bp, Artificial Sequence
  Primer
tcactcactctagacggccgtcgcactcatttac

SEQ ID NO: 3 - DNA, 34 bp, Artificial Sequence
  Primer
gtgagtgagaattcatgcatctcctcggcccctg

SEQ ID NO: 4 - DNA, 37 bp, Artificial Sequence
  Primer
tcactcactcgcggccgcttcgcctgccgatggtctc

(19) United States
(12) Patent Application Publication
(10) Pub. No.: US 2005/0244828 A1
(43) Pub. Date: Nov. 3, 2005

(54) Elisa assay of serum soluble cd22 to assess tumor burnden/relapse in subjects with leukemia and lymphoma

(76) Inventors: Robert J. Kreitman, Potomac, MD (US); Kakushi Matsushita, Kayoshima City (JP); Wyndham Wilson, Washington, DC (US); Ira H. Pastan, Potomac, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/514,910

(22) PCT Filed: May 20, 2003
(86) PCT No.: PCT/US03/16298

§ 371 Date: Nov. 16, 2004

Related U.S. Application Data
(60) Provisional application No. 60/382,269, filed on May 20, 2002.

(51) Int. Cl. C12Q 1/68; G01N 33/574

(52) U.S. Cl. 435/6; 435/7.23

(57) ABSTRACT

Disclosed herein are methods of using previously unknown soluble forms of CD22 (sCD22) present in the serum of subjects with B-cell leukemias and lymphomas to assess tumor burden in the subjects. Also disclosed are methods of diagnosing or prognosing development or progression of a B-cell lymphoma or leukemia in a subject, including detecting sCD22 in a body fluid sample taken or derived from the subject, for instance serum.

### FIELD OF THE DISCLOSURE

The present disclosure is related to diagnosing, prognosing, staging, preventing, and treating disease, particularly B-cell leukemia and lymphoma.

### BACKGROUND

Tumor burden in subjects with lymphoma or leukemia is generally assessed using X-rays, blood tests, and needle biopsies or aspirates of bone narrow. Tumor cells in the bone marrow often make up a considerable percentage of the total tumor burden, but needle biopsies and aspirates of bone marrow are painful and rarely quantitative. Moreover, X-rays and tests involving nuclear medicine often cannot determine whether a residual mass is a tumor, scar tissue, or benign inflammation. Thus, even if a needle biopsy of the mass is positive, it is still possible that most of the mass is benign, making it difficult to determine the true tumor burden of the subject.

Many types of B-cell lymphomas and leukemias are positive for the cell surface antigen CD22, including the majority of non-Hodgkin's lymphoma, acute lymphocytic leukemia, and chronic lymphocytic leukemia. CD22 is a B-cell-restricted, integral membrane glycoprotein, the sequence of which defines it as a member of the immunoglobulin (Ig) superfamily. CD22 functions in B-cell activation and as an adhesion molecule, mediating interactions with activated blood cells and accessory cells (Hanasaki et al., J. Biol. Chem., 270(13):7533-7542, 1995).

There are a few tumor markers for monitoring tumor burden of B-cell malignancies such as thymidine kinase (TK) (Gronowitz et al., British Journal of Cancer 1983, 47:487-495), β2-microglobulin (Amlot et al., The Lancet 1978, 2(8087):476), and soluble CD25 (Ambrosetti et al., International Journal of Clinical Laboratory Research 1993, 7:23:34-37). TK and β2-microglobulin are not particularly sensitive for B-cell malignancies, and soluble CD25 is only elevated in subjects with CD25-positive B-cell lymphoma and leukemia, a small subset of all B-cell lymphomas and leukemias.

### SUMMARY OF THE DISCLOSURE

Disclosed herein is an assay for assessing B-cell leukemia or B-cell lymphoma in a subject using previously unknown soluble forms of CD22 (sCD22) present in biological samples. The assay includes measuring a level of soluble CD22 in a biological sample from the subject, and correlating the level of soluble CD22 with a presence or activity of the B-cell leukemia or B-cell lymphoma. Uses of the assay include, but are not limited to diagnosing a B-cell leukemia or B-cell lymphoma, determining the tumor burden of a B-cell leukemia or B-cell lymphoma, predicting the development of a B-cell leukemia or B-cell lymphoma, determining an appropriate anti-tumor therapy for a subject with a B-cell leukemia or B-cell lymphoma, measuring clinical progression or regression of a B-cell leukemia or B-cell lymphoma, and determining whether to initiate anti-tumor therapy in a subject who does not have other clinical evidence of a B-cell leukemia or B-cell lymphoma.

Other embodiments are kits for measuring a soluble CD22 level, which kits include a specific binding molecule that selectively binds to the CD22, e.g. an antibody or antibody fragment that selectively binds CD22.

Also disclosed are methods for screening for a compound useful in treating, reducing, or preventing B-cell lymphomas or leukemias, or development or progression of B-cell lymphomas or leukemias, which methods include determining if application of a test compound lowers soluble CD22 levels in a subject, and selecting a compound that so lowers sCD22 levels.

Further embodiments are methods of monitoring progress of treatment for a B-cell lymphoma or B-cell leukemia in a subject The methods include administering an anti-tumor compound or putative anti-tumor compound to the subject, and monitoring a soluble CD22 level in the subject to determine whether the soluble CD22 level falls as an indication that the compound is reducing tumor burden in the subject.

Still other embodiments are methods for treating a B-cell lymphoma or B-cell leukemia in a subject. The methods include obtaining a body fluid sample from a subject, identifying an elevated level of soluble CD22 relative to a control, and administering a therapeutically effective amount of an anti-cancer agent, thereby treating the subject or inhibiting the B-cell lymphoma or leukemia.

Yet still other embodiments are methods of diagnosing or prognosing development or progression of a B-cell lymphoma or B-cell leukemia in a subject. These methods include contacting a body fluid sample from the subject with a CD22-specific binding agent, detecting whether the binding agent is bound by the sample, thereby measuring the levels of the soluble CD22 present in the sample, and comparing in the level of sCD22 in the sample to a control value, wherein the control value represents the level of soluble CD22 found an analogous sample from a subject not having a B-cell lymphoma or B-cell leukemia, or a standard soluble CD22 level in analogous samples from a subject not having a B-cell lymphoma or B-cell leukemia or not having a predisposition for developing a B-cell lymphoma or B-cell leukemia, and wherein a sCD22 level greater than the control level is diagnostic or prognostic for development or progression of a B-cell lymphoma or B-cell leukemia in the subject

### BRIEF DESCRIPTION OF THE FIGURES

FIG. 1 is a standard curve for soluble CD22. Purified sCD22 was tested by ELISA assay as described herein.

FIG. 2 is a graph of a BL22 cytotoxicity assay in Raji cells, demonstrating that the sCD22 standard used is capable of binding to BL22 and inhibiting cytotoxic activity toward CD22-positive target cells.

FIG. 3 is a graph showing levels of sCD22 in subjects with hairy cell leukemia (HCL) and chronic lymphocytic leukemia (CLL) prior to treatment, demonstrating that subjects with HCL or CLL exhibit elevated sCD22 levels as compared to normal controls. Soluble CD22 levels were determined by ELISA as described herein.

FIG. 4 is a graph showing levels of sCD22 in subjects with HCL who were undergoing their last therapy cycle. Soluble CD22 levels were determined by ELISA as described herein. Subjects in complete remission (CR) were compared to subjects not achieving complete remission.

FIG. 5 is a series of graphs showing a time course of sCD22 levels in subjects with HCL and CLL. FIG. 5A is a graph showing a time course of sCD22 levels in subjects with HCL who achieved complete remission (CR). FIG. 5B is a graph showing a time course of sCD22 levels in subjects with HCL who did not achieve complete remission. FIG. 5C is a graph showing a time course of sCD22 levels in a subject with CLL who achieved partial remission. Values shown were measured at the beginning of each cycle. The subject had an 11 month delay after cycle 12 before continuing on cycle 13, resulting in progressive disease between treatment cycles 12 and 13.

FIG. 6 is a series of graphs showing the correlation between sCD22 levels and other indicators of disease progression. FIG. 6A is a graph showing the correlation between sCD22 and peripheral malignant cell count in subjects with CLL and HCL. FIG. 6B is a graph showing the correlation between sCD22 and spleen volume in subjects with CLL and HCL. FIG. 6C is a graph showing the correlation between sCD22 and cell surface CD22 sites in subjects with CLL and HCL.

FIG. 7 is a pair of graphs showing the sCD22 dynamics in a group of subjects with previously untreated large B-cell lymphomas. FIG. 7A is plotted on a linear axis, and FIG. 7B is plotted on a logarithmic axis. Six each were HUV negative and positive. Subject demographics included: median (range) age of 43 (25-64) and advanced stage lymphoma in 58%. Serum was analyzed for soluble CD22 levels at the following time points: pre-treatment; during treatment of cycles 3, 4, 5, 6 and 7; and post-treatment. The black arrows show six subjects with disease progression and the red arrows indicate subjects in durable remissions. All subjects showed a decrement in soluble CD22 levels with treatment, and all subjects who relapsed showed an increase in CD22 levels. However, optimal time points for relapsed subjects were not always available.

### BRIEF DESCRIPTION OF THE SEQUENCE LISTING

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NOs: 1 and 2 are human Fc gene-specific primers used for amplification during PCR.

SEQ ID NOs: 3 and 4 are human CD22 gene-specific primers used for amplification during PCR.

### DETAILED DESCRIPTION

I. Abbreviations

BL22 a recombinant immunotoxin containing the FV domains of RFB4 fused to PE38, a truncated form of Pseudomonas exotoxin.
BSA bovine serum albumin
CLL chronic lymphocytic leukemia
DMEM Dulbecco's minimal essential medium
EPOCH EPOCH is a pneumonic standing for the following chemotherapy agents:
   E Etoposide
   P Prednisone
   O Vincristine (oncovine)
   C cyclophosphamide
   H doxorubicin
HCL hairy cell leukemia
RFB4 RFB4(dsFv)-PE38, a mouse MAb against human CD22
sCD22 soluble CD22
   II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In accordance with the present disclosure, conventional molecular biology, microbiology, and recombinant DNA techniques within the skill of the art are used. Such techniques are fully explained in the literature (see, e.g., Sambrook et al., 1989. Molecular cloning, a laboratory manual. 2^{nd} ed. Cold Spring Harbor Laboratory, Cold spring Harbor, N.Y., Glover, 1985, DNA Cloning: A practical approach, volumes I and II oligonucleotide synthesis, MRL Press, LTD., Oxford, U.K.; Hames and Higgins, 1985, Transcription and translation; Hames and Higgins, 1984, Animal Cell Culture; Freshney, 1986, Immobilized Cells And Enzymes, IRL Press; and Perbal, A Practical Guide to Molecular Cloning, John Wiley & Sons, New York, 1988).

In order to facilitate review of the various embodiments, the following explanations of specific terms are provided:

An antibody is a protein (or protein complex) that includes one or more polypeptides substantially encoded by immunoglobulin genes or fragments of immunoglobulin genes. The recognized immunoglobulin genes include the kappa, larnbda, alpha, gamma, delta, epsilon and rnu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

As used herein, the term antibodies includes intact immunoglobulins as well as a number of well-characterized fragments produced by digestion with various peptidases, or genetically engineered “artificial” antibodies. Thus, for example, pepsin digests an antibody below the disulfide linkages in the hinge region to produce F(ab)′₂, a dimer of Fab which itself is a light chain joined to V_{H}-C_{H} 1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially a Fab withpart of the hinge region (see, Fundamental Immunology, W. E. Paul, ed., Raven Press, N.Y., 1993). While various antibody fragments are defined in terms of the digestion of an intact antibody, it will be appreciated that Fab′ fragments may be synthesized de novo either chemically or by utilizing recombinant DNA methodology. Thus, the term antibody as used herein also includes antibody fragments either produced by the modification of whole antibodies or synthesized de novo using recombinant DNA methodologies.

Antibodies for use in the methods and devices of this disclosure can be monoclonal or polyclonal. Merely by way of example, monoclonal antibodies can be prepared from murine hybridomas according to the classical method of Kohler and Milstein (Nature 256:495-497, 1975) or derivative methods thereof. Detailed procedures for monoclonal antibody production are described in Harlow and Lane (Antibodies, A Laboratory Manual, CSHL, New York, 1988).

An antigen is a molecule, or fragment thereof, which can induce an immune response in a mammal. The term includes immunogens and regions responsible for antigenicity or antigenic determinants. A chemical or biochemical structure, determinant, antigen or portion thereof that is capable of inducing the formation of an antibody can be referred to as being “antigenic.” “Antigenic determinant” refers to a region of a specified protein that is recognized by an antibody.

An anti-tumor agent, anti-cancer agent, or antineoplastic is any drug that controls or kills neoplastic cells. An anti-tumor agent is used in chemotherapy as a component of an anti-tumor therapy to kill cancer cells. Anti-tumor agents and drugs used in anti-tumor therapy include, but are not limited to: Altretamine, Asparaginase, BCG, Bleomycin sulfate, Busulfan, Carboplatin, Carmustine, Chlorambucil, cis-platinum, cis-diammine-dichloroplatinum, 2-chlorodeoxyadenosine, Cyclophosphamide, cytosine arabinoside, Dacarbazine imidazole carboxamide, Dactinomycin, Daunorubicin, daunomycin, Dexamethasone, Doxorubicin, Etoposide, epipodophyllotoxin, Floxuridine, Fluorouracil, Fluoxymesterone, Flutamide, Fludarabine, Goserelin, Hydroxyurea, Idarubicin HCL, Ifosfamide, Isophosphamide, Interferon α, Interferon α2α, Interferon α2b, Interferon αn3, Irinotecan, Leucovorin calcium, Leuprolide, Levamisole, Lomustine, Megestrol, Melphalan, L-phenylalanine mustard, Melphalan hydrochloride, MESNA, Mechlorehamine, Methylprednisolone, Methotrexate, Amethopterin, Mitomycin, Mitomycin-C, Mitoxantrone, Mercaptopurine, Paclitaxel, Plicamycin, Mithramycin, Prednisone, Procarbazine, Streptozocin, Tamoxifen, 6-thioguanine, Thiotepa, triethylene thiophosphoramide, Vinblastine, Vincristine, and Vinorelbine tartrate.

Assessing a disease or condition refers to determining a status of the disease or condition. For example, assessing includes, but is not limited to detecting, diagnosing, prognosing, monitoring, and identifying a disease or condition. In some embodiments, assessing includes quantitatively or qualitatively determining the presence of soluble CD22 in a sample.

A B-cell leukemia is any of a group of diseases of the reticuloendothelial system that preferentially affect B-cells and involve uncontrolled proliferation of white blood cells (leukocytes). A B-cell lymphoma is any of a group of malignancies of lymphoid tissue (lymph nodes, spleen, and other organs) that preferentially affects B-cells. B-cell lymphomas and leukemias include, for example: B-cell lymphoblastic leukemia/lymphoma, B-cell chronic lymphocytic leukemia/small lymphocytic lymphoma, lymphoplasmacytic lymphoma, splenic marginal zone B-cell lymphoma, hairy cell leukemia, extranodal marginal zone B-cell lymphoma of the mucosa-associated lymphoid tissue (MALT) type, mantle cell lymphoma, follicular lymphoma, nodal marginal zone lymphoma with or without monocytoid B cells, diffuse large B-cell lymphoma, and Burkitt's lymphoma. B-cell lymphomas and 3-cell leukemias are associated with increased expression of cell-surface CD22, and can be identified, diagnosed, monitored, and prognosed using the methods described herein. For example, B-cell lymphomas and B-cell leukemias can be diagnosed by detecting or monitoring soluble CD22. An activity of a B-cell leukemia or lymphoma includes, but is not limited to loss of differentiation, increased rate of growth, invasion of surrounding tissue, and metastasis A biological sample is a sample obtained from body cells of a subject, such as those present in peripheral blood, cerebrospinal fluid, serum, bone marrow, urine, saliva, tissue biopsy, surgical specimen, amniocentesis samples and autopsy material.

A body fluid is any bodily fluid or secretion of fluid from an animal, for instance as blood, serum, semen, urine, cerebrospinal fluid, or saliva.

A cancer is a biological condition in which a malignant tumor or other neoplasm has undergone characteristic anaplasia with loss of differentiation, increased rate of growth, invasion of surrounding tissue, and/or which is capable of metastasis. A tumor is an abnormal mass of tissue or neoplasm that may be either malignant or non-malignant.

The term cancer includes B-cell lymphomas and leukemias. Also included are different stages of a single cancer, for instance both primary and recurrent B-cell leukemia or lymphoma, and cancer at any progressive stage, such as Stages I-IV.

A subject may be classified into a B-cell leukemia or lymphoma stage based upon evaluation of a biological sample from the subject for indices known in the art or disclosed herein as being indicative of that stage of B-cell leukemia or lymphoma. For example, a subject may be classified as having a cancer state of cancer-free, active cancer (i.e., stage I, II, III, or IV B-cell leukemia or lymphoma), or in remission from previous cancer.

CD22 is a 135 kDa phosphoglycoprotein adhesion molecule present on the surface of B cells, including human B cell lymphomas and leukemias. Soluble forms of CD22 (sCD22) are disclosed herein, and are recognized by antibodies (such as those disclosed herein) that specifically bind to CD22.

Chronic lymphocytic leukemia (CLL) is a lymphoproliferative disorder characterized by lymphocytosis, lymphadenopathy, and organomegaly. It can be conceived of as a lymphoma that involves the peripheral blood.

Clinical progression of a disease or disorder refers to a general worsening of the disease or disorder, or an increase in the likelihood of developing the disease or disorder. For example, clinical progression of a B-cell leukemia or B-cell lymphoma includes an increase in tumor burden or tumor load, the number of cancer cells, the size of a tumor, or the amount of cancer in a body. In some examples, clinical progression of a B-cell leukemia or B-cell lymphoma includes an increase in a level of soluble CD22 relative to the level found in a subject who does not have a known B-cell leukemia or lymphoma, or relative to a level of soluble CD22 in a population of subjects who do not have a known B-cell leukemia or lymphoma, or relative to the sCD22 level in a sample taken at an earlier time. Clinical regression of a disease or disorder refers to a general improvement in the disease or disorder, or to a decrease in the likelihood of developing the disease or disorder. For example, clinical regression of a B-cell leukemia or B-cell lymphoma includes a decrease in tumor burden or tumor load, the number of cancer cells, the size of a tumor, or the amount of cancer in a body. In some examples, clinical regression of a B-cell leukemia or B-cell lymphoma includes a decrease in a level of soluble CD22 relative to the sCD22 level in a sample taken from the subject at an earlier time.

Detecting or detection refers to quantitatively or qualitatively determining the presence of a biomolecule under investigation. For example, detecting a B-cell leukemia or B-cell lymphoma includes quantitatively or qualitatively determining the presence of soluble CD22 in a sample. Monitoring a B-cell lymphoma or B-cell leukemia includes detecting a B-cell lymphoma or B-cell leukemia, or detecting the progression or regression of a B-cell lymphoma or B-cell leukemia. Diagnosing or diagnosis of a B-cell lymphoma or B-cell leukemia includes both detecting a B-cell lymphoma or B-cell leukemia and identifying a B-cell lymphoma or B-cell leukemia, for example identifying a B-cell leukemia as hairy cell leukemia or chronic lymphocytic leukemia.

Hairy cell leukemia (HCL) is a malignant disorder of small B-lymphocytes that gets its name from the presence of cytoplasmic projections in these cells. Subjects with HCL commonly present with pancytopenia, splenomegaly and marrow fibrosis. The peripheral blood usually contains a small number of hairy cells, but it is uncommon to have a “leukemic picture”. Hairy cells proliferate in the red pulp of the spleen, so splenomegaly is common.

A label is any molecule or composition that is detectable by, for instance, spectroscopic, photochemical, biochemical, immunochemical, electrical, optical, or chemical means. Examples of labels, including radioactive isotopes, enzyme substrates, co-factors, ligands, chemiluminescent or fluorescent agents, haptens, enzymes, colloidal gold particles, colored latex particles, and epitope tags, have been disclosed previously and are known to those of ordinary skill (see, for instance, U.S. Pat. Nos. 4,275,149; 4,313,734; 4,373,932; and 4,954,452).

The attachment of a compound (e.g., an antibody) to a label can be through covalent bonds, adsorption processes, hydrophobic and/or electrostatic bonds, as in chelates and the like, or combinations of these bonds and interactions and/or may involve a linking group. A labeled antibody or specific binding agent can bind to a target to produce a detectable complex. Such an antibody or agent is detectably labeled.

Primers are short nucleic acids, preferably DNA oligonucleotides 10 nucleotides or more in length, which are annealed to a complementary target DNA strand by nucleic acid hybridization to form a hybrid between the primer and the target DNA strand, then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Primers as used in the present disclosure preferably comprise at least 10 nucleotides of the nucleic acid sequences that are shown to encode specific proteins. In order to enhance specificity, longer primers may also be employed, such as primers that comprise 15, 20, 30, 40, 50, 60, 70, 80, 90 or 100 consecutive nucleotides of the disclosed nucleic acid sequences. Methods for preparing and using probes and primers are described in the references, for example Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences; Innis et al. (1990) PCR Protocols, A Guide to Methods and Applications, Innis et al. (Eds.), Academic Press, San Diego, Calif. PCR primer pairs can be derived from a known sequence, for example, by using computer programs intended for that purpose such as Primer (Version 0.5, 1991, whitehead Institute for Biomedical Research, Cambridge, Mass.).

When referring to a primer, the term specific for (a target sequence) indicates that the primer hybridizes under stringent conditions substantially only to the target sequence in a given sample comprising the target sequence.

Prognosis or prognosing refers to a prediction of the probable course and outcome of a disease, or the likelihood of developing a disease. A prognosis can include a prediction of the likelihood of recovery from a disease, or a prediction of the likelihood of developing a disease. In some examples, a prognosis of a B-cell leukemia or B-cell lymphoma includes a determination of tumor burden or tumor load, the number of cancer cells, the size of a tumor, or the amount of cancer in a body. In some examples, a prognosis of a B-cell leukemia or B-cell lymphoma includes a determination of a level of soluble CD22 relative to the level found in a subject who does not have a known B-cell leukemia or lymphoma, or relative to a level of soluble CD22 in a population of subjects who do not have a known B-cell leukemia or lymphoma, or who have a particular disease stage, or relative to the sCD22 level in a sample taken from the subject at an earlier time. Generally, an increase one or more of these factors indicates a worsening or progression of the disease, whereas a decrease in one or more of these factors indicates a regression of disease, for example a partial or total remission. In some embodiments, the level of sCD22 found in a subject who does not have a known B-cell leukemia or lymphoma, or in a population of subjects who do not have a known B-cell leukemia or lymphoma, is referred to as a normal value. Tumor burden, tumor load, the number of cancer cells, the size of a tumor, or the amount of cancer in a body, and soluble CD22 levels provide clinical evidence of a B-cell Leukemia or lymphoma.

A protein is a biological molecule expressed by a gene and comprised of amino acids.

A purified molecule is one that has been purified relative to its original environment. The term “purified” does not require absolute purity; rather, it is intended as a relative term. Thus, for example, a purified protein preparation is one in which the protein referred to is more pure than the protein in its natural environment within a cell or within a production reaction chamber (as appropriate).

Soluble CD22 (sCD22) is a non-membrane-bound form of CD22, a 135 kDa phosphoglycoprotein adhesion molecule present on the surface of B cells, including human B cell lymphomas and leukemias (Genbank Accession No. XM-009320). Soluble CD22 can be any portion of the CD22 protein not connected to the membrane, and is usually a truncated form of CD22. For example, sCD22 can be about 100 kDa, however it can also be no more than or no less than about 90, 80, 70, 60, 50, 40, or 30 kDa, or smaller. Standard software is available for determining the transmembrane domain of CD22.

A specific binding agent is an agent that binds substantially only to a defined target. Thus a protein-specific binding agent binds substantially only the specified protein. The term “protein specific binding agent” includes anti-protein antibodies (and functional fragments thereof) and other agents (such as soluble receptors) that bind substantially only to the specified protein. In some embodiments, a specific binding agent is conjugated to s detectable label or moiety, for example an alkaline phosphatase, horseradish peroxidase, or a radioactive or fluorescent tag. The binding of such a labeled specific binding agent results in a detectable complex.

Anti-protein antibodies (such as anti-CD22 antibodies) may be produced using standard procedures described in a number of texts, including Harlow and Lane (Antibodies, A Laboratory Manual, CSHL, New York, 1988). The determination that a particular agent binds substantially only to the specified protein, or component epitopes thereof, may readily be made by using or adapting routine procedures. One suitable in vitro assay makes use of the Western blotting procedure (described in many standard texts, including Harlow and Lane (Antibodies, A Laboratory Manual, CSHL, New York, 1988)). Western blotting may be used to determine that a given protein binding agent, such as an anti-Acr monoclonal antibody, binds substantially only to the specified protein.

Shorter fragments of antibodies can also serve as specific binding agents. For instance, Fabs, Fvs, and single-chain Fvs (SCFvs) that bind to Acr would be Acr-specific binding agents. These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Methods of making these fragments are routine.

A subject is a living, multi-cellular vertebrate organism a category that includes both human and non-human mammals.

A therapeutic agent, as used in a generic sense, includes treating agents, prophylactic agents, and replacement agents. A therapeutically effective amount of a compound or drug is a dose sufficient to prevent advancement, or to cause regression of the disease, or which is capable of relieving symptoms caused by the disease, such as pain or swelling.

A tumor is a neoplasm that may be either malignant or non-malignant, and includes both solid and non-solid tumors (such as hematologic malignancies). Tumor burden or tumor load refers to the number of cancer cells, the size of a tumor, or the amount of cancer in the body. Generally, an increase in tumor burden indicates a worsening or progression of disease, whereas a decrease in tumor burden indicates a regression of disease, for example a partial or total remission. Tumor burden is one factor used to determine a disease prognosis.

Unless otherwise explained, all technical and scientific terms used herein have the same meaning as commonly understood by one of ordinary skill in the art to which this disclosure belongs. The singular terms “a,” “an,” and “the” include plurals unless the context clearly indicates otherwise. Similarly, the word “or” is intended to include “and” unless the context clearly indicates otherwise. “Comprises” means “includes.” Hence, “comprises A or B” means including A, B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

III. Description of Several Specific Embodiments

Disclosed herein are assays for assessing B-cell leukemia or B-cell lymphoma in a subject. The assays include measuring a level of soluble CD22 in a biological sample from the subject, and correlating the level of soluble CD22 with a presence or activity of the B-cell leukemia or B-cell lymphoma. These assays are useful for a variety of purposes, including, but not limited to diagnosing a B-ell leukemia or B-cell lymphoma, determining the tumor burden of a B-cell leukemia or B-cell lymphoma, predicting the development of a B-cell leukemia or B-cell lymphoma, determining an appropriate anti-tumor therapy for a subject with a B-cell leukemia or B-cell lymphoma, measuring clinical progression or regression of a B-cell leukemia or B-cell lymphoma, and determining whether to initiate anti-tumor therapy in a subject who does not have other clinical evidence of a B-cell leukemia or B-cell lymphoma.

In some embodiments, the assay is used to diagnose a B-cell leukemia or B-cell lymphoma by determining whether the soluble CD22 level is elevated above a predetermined level associated with the presence of B-cell leukemia or B-ell lymphoma. In other embodiments, the assay is useful for determining tumor burden of the B-cell leukemia or B-cell lymphoma, and is carried out by detecting a change in the level of soluble CD22, wherein an increase in the level of soluble CD22 indicates an increase in a tumor burden of the B-cell leukemia or B-cell lymphoma and a decrease in the level of the soluble CD22 indicates a decrease in the tumor burden of the B-cell leukemia or B-cell lymphoma.

In some embodiments, the assay is an assay for predicting development of the B-cell leukemia or B-cell lymphoma in the subject, and an elevation in the level of the soluble CD22 above a predetermined level indicates an increased likelihood of the subject developing B-cell leukemia or B-cell lymphoma. In some examples, measuring a level of soluble CD22 involves making multiple measurements of the level of soluble CD22; and wherein a change in the level of the soluble CD22 is an indication of changing tumor burden in the subject. In particular examples, the B-cell leukemia or B-cell lymphoma is hairy cell leukemia or chronic lymphocytic leukemia. In more particular examples, the predetermined value is a normal value determined in a population of subjects who do not have a known B-cell leukemia or B-cell lymphoma.

In certain examples of the assay, the biological sample is a serum sample. In certain other examples, measuring a level of soluble CD22 in a biological sample involves contacting the biological sample with a specific binding agent to form a detectable complex, and quantitating the detectable complex. In particular examples of the assay, the specific binding agent is detectably labeled.

In further embodiments of the assay, the assay is an assay for determining an appropriate anti-tumor therapy for the subject. In certain examples of the embodiment, the anti-tumor therapy is administered to the subject, and the anti-tumor therapy is continued if the level of soluble CD22 subsequently declines. In still further embodiments of the assay, the assay comprises an assay for identifying an anti-tumor agent for treatment of B-cell leukemia or B-cell lymphoma. Certain examples of these embodiments include administering a test agent to the subject and subsequently determining whether the level of soluble CD22 declines, wherein a decline in the level of soluble CD22 indicates the test agent is an anti-tumor agent.

Still further embodiments of the assay are used for measuring clinical progression of B-cell leukemia or B-cell lymphoma in a subject known to have B-cell leukemia or B-cell lymphoma. In certain examples of the embodiments, an increase in the level of soluble CD22 indicates an increase in tumor burden of the B-cell leukemia or B-cell lymphoma. Still other embodiments of the assay are used for measuring clinical regression of B-cell leukemia or B-cell lymphoma in a subject known to have B-cell leukemia or B-cell lymphoma. In certain examples of these embodiments, a decrease in the level of soluble CD22 indicates a decrease in tumor burden of the B-cell leukemia or B-cell lymphoma. Still further embodiments of the assay are used for determining whether to initiate anti-tumor therapy in a subject who does not have other clinical evidence of B-cell leukemia or B-cell lymphoma. In certain examples, the anti-tumor therapy is administered to the subject if the level of soluble CD22 is above a predetermined value associated with a predisposition to develop B-cell leukemia or B-cell lymphoma.

Also disclosed are kits for measuring a soluble CD22 level, comprising a specific binding agent that selectively binds to the CD22. In some embodiments, the binding molecule is an antibody or antibody fragment that selectively binds CD22.

Further embodiments are methods of testing a compound to determine whether it is useful in treating, reducing, or preventing B-cell lymphomas or B-cell leukemias or development or progression of B-cell lymphomas or B-cell leukemias. The method includes determining if administration of a test compound lowers soluble CD22 levels in a subject, and selecting a compound that so lowers soluble CD22 levels.

Other embodiments are methods of monitoring progress of treatment for a B-cell lymphoma or B-cell leukemia in a subject. The methods include administering an anti-tumor compound or putative anti-tumor compound to the subject, and monitoring a soluble CD22 level in the subject to determine whether the soluble CD22 level falls as an indication that the compound is reducing tumor burden in the subject.

Still further embodiments are methods for treating a B-cell lymphoma or B-cell leukemia in a subject. These methods include obtaining a body fluid sample from a subject, identifying an elevated level of soluble CD22 relative to a control, and administering a therapeutically effective amount of an anti-cancer agent, thereby treating the subject or inhibiting the B-cell lymphoma or leukemia.

Further embodiments are methods of diagnosing or prognosing development or progression of a B-cell lymphoma or B-cell leukemia in a subject. These methods include contacting a body fluid sample from the subject with a CD22-specific binding agent, detecting whether the binding agent is bound by the sample, thereby measuring the levels of the soluble CD22 present in the sample, and comparing in the level of sCD22 in the sample to a control value. In certain examples, the control value represents the level of soluble CD22 found an analogous sample from a subject not having a B-cell lymphoma or B-cell leukemia, or a standard soluble CD22 level in analogous samples from a subject not having a B-cell lymphoma or B-cell leukemia or not having a predisposition for developing a B-cell lymphoma or B-cell leukemia, wherein a sCD22 level greater than the control level is diagnostic or prognostic for development or progression of a B-cell lymphoma or B-cell leukemia in the subject

IV. Soluble CD22

CD22 is a 135 kDa phosphoglycoprotein adhesion molecule present on the surface of B cells, including human B cell lymphomas and leukemias (Clark, Journal of Immunology 1993, 150:4715-4718). CD22 belongs to an Ig subfamily known as the siglecs (sialic acid-binding Ig-like lectins). Ten siglec family members are known currently: sialoadhesin (siglec-1), CD22 (siglec-2), CD33 (siglec-3), myelin-associated glycoprotein (MAG, siglec-4), and siglecs-5 through -10. Siglecs are characterized by an N-terminal V-set Ig-like domain (which mediates sialic acid binding), varying numbers of C2-set Ig-like domains, a transmembrane domain, and an intracellular domain. Because of their transmembrane domains, siglecs are membrane-bound proteins.

It has now been found that a previously unknown, soluble form of CD22 can be detected and quantified in body fluid samples, such as serum. Tumor burden in subjects with B-cell lymphoma or leukemia is generally assessed using X-rays and needle biopsies or aspirates of bone marrow. However, tumor cells in the bone marrow often make up a considerable percentage of the total tumor burden, and needle biopsies and aspirates of bone marrow are painful and rarely quantitative. Moreover, X-rays and tests involving nuclear medicine often fail to determine whether a residual mass is a tumor, scar tissue, or benign inflammation. Thus, even if a needle biopsy of the mass is positive, it is still possible that most of the mass is benign, making it difficult to determine the true tumor burden of the subject. Assessment of tumor burden by measuring sCD22 levels in serum overcomes many of these problems because the test is non-invasive and provides an unambiguous, quantitative determination of a subject's total or relative tumor burden.

Western blot analysis of the previously unknown, soluble CD22 demonstrates that the major species of sCD22 protein are about 100 kDa, which is smaller than the reported size of the membrane-bound CD22 antigen (135 kDa). Thus, without being bound by theory, it is believed that the sCD22 is produced as a truncated form of the surface CD22 antigen, perhaps by proteolytic digestion of the full-length protein.

V. Altered sCD22 Levels in Subjects with B-cell Lymphomas and Leukemias

Levels of sCD22 are elevated in subjects with CLL and HCL, as compared to sCD22 levels in healthy individuals. In addition, levels of sCD22 correlate well to tumor burden in subjects with CLL and HCL, and sCD22 levels decrease with effective cancer therapies. Because of these characteristics, sCD22 can be used to assess tumor burden in subjects with B-cell leukemias and lymphomas.

In general, relative levels of sCD22 in serum samples can be measured against reference serum baselines from subjects free from B-cell lymphomas and leukemias in order to provide a framework for determining normal CD22 levels versus elevated CD22 levels. Thus, an elevated sCD22 level in a subject as compared to a control indicates the presence of a B-cell lymphoma or leukemia.

Additionally, sCD22 levels can be monitored periodically in subjects with HCL or CLL as they undergo successive rounds of cancer therapy. A lowered (or lowering) sCD22 level is an indicator of therapeutic effectiveness. Conversely, elevated sCD22 indicates that a particular therapeutic intervention is ineffective at treating the cancer. Similarly, the absence of a decline in sCD22 is also an indication that tumor burden is not declining in response to this therapy. Thus, sCD22 levels can be used to make decisions regarding choice of therapy, as well as to diagnose or prognose development or progression of a B-cell lymphoma or leukemia.

Soluble CD22 levels are also useful as for the screening of new anti-cancer therapeutic compounds. For instance, potential therapeutic compounds are administered to a subject with a B-cell lymphoma or leukemia, and the therapeutic efficacy of the compounds is ascertained by monitoring sCD22 levels in the subject over time. Efficacy is determined as discussed above for treatments.

VI. Monitoring of sCD22 Levels

Soluble CD22 can be detected in any bodily fluid or secretion of fluid from an animal, for example blood, serum, semen, urine, cerebrospinal fluid, or saliva. In some embodiments, the fluid is a cell-free sample, however the inclusion of cells in a body fluid sample does not preclude the detection and/or quantification of sCD22. In particular examples, the fluid is serum. Soluble CD22 can be detected using known immunological techniques. The presence of sCD22 (or a sCD22 fragment) above a basal level (e.g., a level normally found in a subject known not to be suffering from a B-cell lymphoma or leukemia) in a body fluid sample taken or derived from a subject indicates that the subject suffers from a B-cell lymphoma or leukemia. In addition, the level of sCD22 present in a body fluid sample taken or derived from a subject correlates directly with tumor burden, in that more sCD22 is indicative of a greater tumor burden in that subject.

In some examples, a level of sCD22 is compared to a “normal” level. Such a normal level can be assigned to the level of sCD22 in a sample from an individual who is not known to have a B-cell lymphoma or leukemia, or can be assigned based on a mean level of sCD22 found in a population. For example, a normal level can be determined by measuring the sCD22 level in a statistically significant number of individuals who do not have a known B-cell lymphoma or leukemia. In other examples, such a population study can be used to assess tumor burden or disease stage by correlating sCD22 levels in populations of individuals with a particular known tumor burden or disease stage. In addition, a normal value may be assigned based on the desired sensitivity and specificity of the assay.

Many techniques are known for the detection and quantification of antigen, such as protein or protein fragments. Examples of methods for the detection of antigens in biological samples, including methods employing dip strips or other immobilized assay devices, are disclosed, for instance in the following patents: U.S. Pat. No. 5,965,356 (Herpes simplex virus type seroassay); U.S. Pat. No. 6,114,179 (Method and test kit for detection of antigens and/or antibodies); and U.S. Pat. No. 6,057,097 (Marker for pathologies comprising an autoimmune reaction and/or inflammatory disease). These methods could readily be adapted for detection of sCD22.

By way of example, Western blot analysis can be used to detect and quantify sCD22 in a body fluid sample. In a typical Western blot, proteins are electrophoretically separated on an acrylamide gel, then transferred to a membrane and detected with one or more antibodies. The antibody detection may be direct or indirect For direct antibody visualization of the sCD22 protein, the blot membrane is incubated with a labeled, CD22-specific binding agent, for example an anti-CD22 antibody conjugated to alkaline phosphatase or horseradish peroxidase. For indirect antibody visualization of the sCD22 protein, the blot membrane is incubated first with an unconjugated CD22-specific antibody (primary antibody), then with a labeled antibody (secondary antibody) that recognizes the primary antibody. For instance, secondary antibodies for the indirect detection of primary antibodies are often conjugated with a detectable moiety, such as horseradish peroxidase, alkaline phosphatase, or radioactive or fluorescent tags.

Alternatively, a sandwich ELISA assay can be used to detect and quantify the sCD22. A typical sandwich ELISA format involves a specific immobilized capture antibody, sample, a labeled detection antibody, chromogens, and stop solution. Antigen will bind to the immobilized capture antibody and thus can be detected with one or more antibodies. The antibody detection technique used with an ELISA may be direct or indirect. For direct antibody visualization of the sCD22 protein, anti-CD22 antibody is attached to a substrate, the substrate is incubated with a body fluid sample, and the substrate is then incubated with another anti-CD22 antibody that has been enzyme-conjugated, for example an anti-CD22 antibody conjugated to alkaline phosphatase or horseradish peroxidase. For indirect antibody visualization of the sCD22 protein, anti-CD22 antibody is attached to the substrate, and the substrate is incubated with a body fluid sample. The substrate is then incubated with an unconjugated CD22-specific antibody (primary antibody), then with an enzyme-conjugated antibody (secondary antibody) that recognizes the primary antibody. Secondary antibodies for the indirect detection of primary antibodies are often conjugated with horseradish peroxidase or alkaline phosphatase. A substrate solution is then added, acted upon by the enzyme, and effects a color change. The intensity of the color change is proportional to the amount of antigen in the original sample. Primary and secondary antibodies also can be coupled to radioactive or fluorescent tags. The intensity of radioactive or fluorescent labeling is proportional to the amount of antigen present in the original sample.

Optionally, a microsphere assay (also called flow beads assay) can be used to detect sCD22 in biological fluids (such as a serum sample from a subject). This technology, as represented by systems developed by Luminex Corporation (Austin, Tex.) and other systems developed by Becton Dickinson (Franklin Lakes, N.J.), allows one to process a very small amount of sample, typically 20 μl, to detect a protein, such as sCD22. The principle of this assay is based on the coupling of a capture antibody to microspheres containing specific amounts of, for instance, a red dye and an infrared dye. After incubation of the microspheres with the sample, a secondary detection antibody coupled with biotin and streptavidin coupled with phycoerythrin, the beads are analyzed with a flow cytometer. One laser detects the beads and a second one detects the intensity of the phycoerythrin bound to those beads (see technical notes available from Luminex Corp., for instance at their website or through their catalog).

VII. Anti-CD22 Antibodies

Two anti-CD22 antibodies were used to detect and quantify sCD22 levels in experiments disclosed herein: BL22 (Kreitman et al., New. Engl. J. Med. 345: 241-247, 2001; Kreitman et al., Clin. Cancer Res. 6:1476-1487, 2000; Kreitman et al., Int. J. Cancer. 81: 148-155, 1999) and RFB4 (Amlot et al., Blood. 82: 2624-2633, 1993, Ghetie et al., Cancer Res. 48: 2610-2617, 1988), although other monoclonal or polyclonal antibodies may be produced that specifically bind to CD22, sCD22, or to specific epitopes within the soluble portion of the protein. For example, any of various commercially-available antibodies to CD22 can be used, such as RDI-M1391clb, RDI-M1437clb, RDI-M1727clb, RDI-CBL147, RDI-CBL147FT, RDI-M1391clb, or RDI-CBL147PE, all available from Research Diagnostics Inc. (Flanders N.J.).

Optimally, antibodies raised against CD22 or sCD22 would specifically detect that protein. That is, such antibodies would recognize and bind the CD22 or sCD22 protein and would not substantially recognize or bind to other proteins found in a biological sample. The determination that an antibody specifically detects its target protein is made by any one of a number of standard immunoassay methods; for instance, the Western blotting technique (Sambrook et al., In Molecular Cloning: A Laboratory Manual, CSHL, New York, 1989).

To determine that a given antibody preparation (such as one produced in a mouse or rabbit) specifically detects the target protein by Western blotting, total cellular protein is extracted from B-cell lymphoma and/or leukemia cells, and electrophoresed on a sodium dodecyl sulfate-polyacrylamide gel. The proteins are then transferred to a membrane (for example, nitrocellulose) by Western blotting, and the test antibody preparation is incubated with the membrane. After washing the membrane to remove non-specifically bound antibodies, the presence of specifically bound antibodies is detected by the use of an anti-mouse or anti-rabbit antibody conjugated to an enzyme such as alkaline phosphatase.

Application of an alkaline phosphatase substrate 5-bromo-4-chloro-3-indolyl phosphate/nitro blue tetrazolium results in the production of a dense blue compound by immunolocalized alkaline phosphatase. Antibodies that specifically detect the target CD22 or sCD22 protein will, by this technique, be shown to bind to the target CD22 or sCD22 protein band (which will be localized at a given position on the gel determined by its molecular weight). Non-specific binding of the antibody to other proteins may occur and may be detectable as a weak signal on the Western blot. The non-specific nature of this binding will be recognized by one skilled in the art by the weak signal obtained on the Western blot relative to the strong primary signal arising from the specific antibody-CD22 protein binding.

A. Monoclonal Antibody Production by Hybridoma Fusion

Monoclonal antibody to epitopes of sCD22 protein can be prepared from murine hybridomas according to the classical method of Kohler and Milstein (Nature 256:495, 1975) or derivative methods thereof. Briefly, a mouse is repetitively inoculated with a few micrograms of the selected protein over a period of a few weeks. The mouse is then sacrificed, and the antibody-producing cells of the spleen are isolated. The spleen cells are fused by means of polyethylene glycol with mouse myeloma cells, and the excess un-fused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Enzymol. 70:419, 1980), and derivative methods thereof. Selected positive clones can be expanded and their monoclonal antibody product harvested for use. Detailed procedures for monoclonal antibody production are described in Harlow and Lane (Antibodies, A Laboratory Manual, CSHL, New York, 1988).

B. Polyclonal Antibody Production by Immunization

Polyclonal antiserum containing antibodies to heterogeneous epitopes of a single protein can be prepared by immunizing suitable animal with the expressed protein, which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with either inadequate or excessive doses of antigen resulting in low titer antisera. Small doses (ng level) of antigen administered at multiple intradermal sites appear to be most reliable. An effective immunization protocol for rabbits can be found in Vaitukaitis et al. (J. Clin. Endocrinol. Metab. 33:988-991, 1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony et al. (In Handbook of Experimental Immunology, Wier, D. (ed.) chapter 19. Blackwell, 1973). Plateau concentration of antibody is usually in the range of about 0.1 to 0.2 mg/ml of serum (about 12 μM). Affinity of the antisera for the antigen is determined by preparing competitive binding curves, as described, for example, by Fisher (Manual of Clinical Immunology, Ch. 42, 1980).

C. Antibodies Raised against Synthetic Peptides

A third approach to raising antibodies against sCD22 protein is to use synthetic peptides synthesized on a commercially available peptide synthesizer based upon the predicted amino acid sequence of sCD22.

By way of example only, polyclonal antibodies to specific peptides within sCD22 can be generated through well-known techniques by injecting rabbits with chemically synthesized peptide.

D. Antibodies Raised by Injection of CD22 Protein-Encoding Sequence

Antibodies may be raised against CD22 or sCD22 by subcutaneous injection of a DNA vector that expresses CD22 or sCD22 protein into laboratory animals, such as mice. Delivery of the recombinant vector into the animals may be achieved using a hand-held form of the Biolistic system (Sanford et al., Particulate Sci. Technol. 5:27-37, 1987) as described by Tang et al. (Nature 356:152-154, 1992). Expression vectors suitable for this purpose may include those that express the CD22 or sCD22 encoding sequence under the transcriptional control of either the human β-actin promoter or the cytomegalovirus (CMV) promoter.

Antibody preparations prepared against a CD22 antigen or epitope of such are useful in quantitative immunoassays that determine concentrations of antigen-bearing substances in biological samples; they are also used semi-quantitatively or qualitatively to identify the presence of antigen in a biological sample.

VIII. Kits for Measuring the Level of sCD22 in Body Fluid Samples

Anti-CD22 antibodies can be supplied in the form of a kit for use in the methods provided herein, for instance in detection or monitoring B-cell lymphoma or leukemia in a subject. In such a kit, one or more anti-CD22, or more particularly anti-sCD22 antibodies are provided in one or more containers. The kit may also contain reagents for use in preparing a body fluid sample taken or derived from a subject for screening with the kit The container(s) in which the reagent(s) are supplied can be any conventional container that is capable of holding the supplied form, for instance, plastic boxes, microfuge tubes, ampoules, or bottles. In some applications, negative controls obtained from a subject free from B-cell lymphomas or leukemias is provided in pre-measured (e.g., single use) amounts in individual, typically disposable, tubes or equivalent containers. With such an arrangement, the sample to be tested for the presence of B-cell lymphoma or leukemia can be added to the testing container and tested directly.

The amount/number of each testing reagent and container supplied in the kit can be any appropriate amount, depending for instance on the market to which the product is directed. For instance, if the kit is adapted for research or clinical use, the amount of each testing reagent and container provided would likely be an amount sufficient to screen several biological samples. Those of ordinary skill in the art know the amount of testing reagent that is appropriate for use in a single container. General guidelines may for instance be found in Innis et al. (PCR Protocols, A Guide to Methods and Applications, Academic Press, Inc., San Diego, Calif., 1990), Sambrook et al. (In Molecular Cloning: A Laboratory Manual, Cold Spring Harbor, N.Y., 1989), and Ausubel et al. (In Current Protocols in Molecular Biology, Greene Publ. Assoc. and Wiley-Intersciences, 1992).

Certain embodiments of the disclosure are illustrated by the following non-limiting Examples.

### EXAMPLES

### Example 1

### Determining Tumor Burden in a Subject with a B-Cell Lymphoma or Leukemia

This example demonstrates that serum sCD22 levels can be used to assess tumor burden in subjects with B-cell lymphomas and leukemias. Soluble CD22 levels correlate well with other measures of tumor burden, and can be used as an indicator of complete remission, partial remission, and relapse.

Subjects

Thirty-nine subjects were examined, 16 of whom had previously been diagnosed with CLL and 23 of whom had been diagnosed with CLL. Of the 39 subjects, 10 CLL subjects and 23 HCL subjects received BL22 treatment. Plasma samples were drawn before each cycle of treatment where the immunotoxin, BL22, was administered every other day for 3 doses. Plasma samples were also drawn at additional post-therapy time points. Samples were stored at −80° C. until the measurement.

Therapy Protocol

Between 0.2 and 4 mg of BL22 (a recombinant immunotoxin containing the FV domains of RFB4 fused to PE38, a truncated form of Pseudomonas exotoxin, produced by the Developmental Therapeutics Program of the National Cancer Institute) was diluted in 50 ml of 0.2% serum albumin in 0.9% sodium chloride and administered as a 30-minute infusion every other day for a total of three doses. Subjects without neutralizing antibody, who did not have progressive disease, were treated again after restaging at intervals of three weeks or more. Subjects with complete remission received two more therapy cycles of RFB4 (the mouse MAb against human CD22) as a consolidation therapy. A total of 121 plasma pre-therapy cycle samples from 39 subjects were drawn for sCD22 assay.

Verification of Tumor Burden

To assess the correlation of levels of soluble CD22 to total tumor burden, the total numbers of cell surface CD22 antigen were calculated using numbers of CD22 sites/cell, peripheral blood malignant cell counts determined by lymphocyte counts and flow cytometry analysis, and the volumes of spleen. Spleen volumes were calculated using the formula:
Spleen volume(cm³)=30+0.58×[height (cm)×width(cm)×length(cm)]−214.6 cm³
(Pasopoulos et al., European Radiology 1997, 7:246-248).

To eliminate contaminated normal spleen tissue, the mean value of healthy volunteers (214.6 cm³) was subtracted. Since malignant cells usually shared spleens, the estimated malignant cell number was 10⁹/ml. Taken together, the total number of CD22 antigen was calculated as follows:
Surface CD22×total malignant cell count[total counts of peripheral blood+106×volume of spleen(cm³)].

Tumor burden was also assessed by peripheral blood lymphocyte count and flow cytometry to detect CLL and HCL antigens, and was computed tomographically.

The criteria for complete remission were: an absence of evidence of disease in radiographic study and an absence of tumor cells in the bone marrow and peripheral blood according to morphological criteria, as ascertained at least four weeks after the last dose of BL22. The criteria for partial response was reduction in tumor burden by at least 50% assessed at least 28 days after the last dose of BL22.

In addition, cell surface sites of CD22 antigen were measured by binding assay as described previously (Robbins et al., Clinical Cancer Research, 2000, 6:693-700). Briefly, cells from subjects were washed and resuspended in binding buffer (DMEM containing 0.2% sodium azide and 0.1% BSA), and added in 0.15 ml aliquots to 96 well U-bottomed plates. Among the subjects studied, the number of cells/well ranged from 2×10⁶ to 1×10⁷. Varying amounts of [¹²⁵I]-labeled anti-CD22 (RFB4) were added to the cells in 0.05 ml aliquots. After 45-90 minutes of incubation at 4° C., the plate was centrifuged, and cells were washed by cold binding buffer, the radioactivity associated with the resuspended cells was counted, and the numbers of sites/cell were calculated using Scatchard plots. The correlation between sCD22 levels and other parameters is shown in Table 1.

TABLE 1
Correlation between sCD22 levels and parameters

| Parameters | n | relation | z value | p value |
|---|---|---|---|---|
| Lymphocytes | 136 | 0.386 | 4.693 | <0.0001 |
| Spleen | 121 | 0.401 | 4.615 | <0.0001 |
| Malignant cells | 115 | 0.433 | 4.905 | <0.0001 |
| CD22 sites | 56 | 0.452 | 3.551 | 0.0004 |

Preparation and Purification of CD22 Extracellular Domain

The extra-cellular domain of CD22 protein was expressed as a fusion protein to human IgG Fc in transfected 293T cells. The human Fc fragment was amplified using PCR from plasmid Ret-Fc (provided by M. Billaud) with primers coding: 5′-GAGTGAGTGCGGCCGCGGTGGTCGTCGTGCATCCGT-3′ (SEQ ID NO. 1) and noncoding: 5′-TCACTCACTCTAGACGGCCGTCGCACTCATITAC-3′ (SEQ ID No. 2) introducing 5′ Nod and 3′ XbaI restriction sites. After digestion with NotI and XbaI, the PCR product was purified and cloned into the multiple cloning site of vector pCDNA1.1 between NotI and XbaI sites creating plasmid pCDNA1.1-Fc. The extra-cellular domain of CD22 was cloned into pCDNA1.1-Fc creating an in-frame fusion with the Fc. CD22 was amplified from plasmid pRKm22 using the following oligomers: 22 coding: 5′-GTGAGTGAGAATTCATGCATCCCCCGGCCCCTG-3′ (SEQ ID NO. 3) and 33 non-coding: 5′-TCACTCACTCGCGGCCGCTTCGCCTGCCGATGGTCTC-3′ (SEQ ID NO. 4). PRKm22 is a plasmid encoding full-length human CD22 (Sausville et al., Blood 1995, 85:3457-3465) obtained by cloning from a Daudi cDNA Quick clone library (Clontech). The oligomers introduced EcoRI and NotI restriction sites. After digestion with NotI and EcoRI, the PCR product was purified and cloned into vector pCDNA1.1-Fc between NotI and EcoRI sites creating plasmid pCDNA1.1-22-Fc. The 293 T cells were transfected with plasmid pCDNA1.1-22-Fc by lipotransfection using lipotransfectamine (Gibco BRL). Culture supernatant was collected after 48 hrs protein-free culture using Ultradoma (BioWhitaker) and concentrated. Then the CD22-Fc fusion protein was purified with sizing column.

The CD22-Fc fusion protein was added to a BL22 cytotoxicity assay (see below) against the B-cell line, Raji, to determine the biological function. Briefly, 1×10⁴/well Raji cells were cultured in 10% fetal bovine serum (FBS) RPMI1640 medium for 24 hrs in 96 well plate with varying doses of BL22 and sCD22-Fc fusion protein. The culture was pulsed with [³H]-leucine and harvested by a cell harvester. Protein synthesis was measured using a gamma counter.

Cytotoxicity of BL22 in Raji Cells

The cytotoxicity assay is a functional assay demonstrating that the sCD22 standard used is capable of binding to BL22 and inhibiting its cytotoxic activity toward CD22+ target cells.

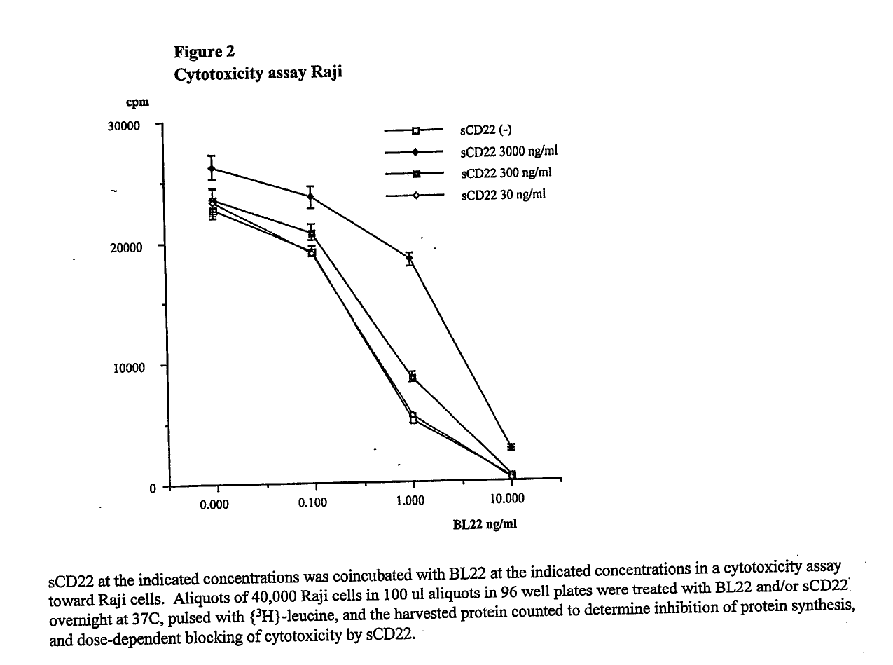

Soluble CD22 was coincubated at the indicated concentrations with BL22 in a cytotoxicity assay toward Raji cells. Aliquots of 40,000 Raji cells in 100 aliquots in 96 well plates were treated with BL22 and/or sCD22 overnight at 37° C., pulsed with [³H]-leucine, and the harvested protein counted to determine inhibition of protein synthesis and dose-dependent blocking of cytotoxicity by sCD22. FIG. 2 shows the inhibition of BL22 cytotoxicity against Raji cells by sCD22-Fc fusion protein. At higher concentrations (300 ng/ml or more), sCD22-Fc fusion protein could inhibit the cytotoxic activity.

ELISA System for Measuring sCD22

The following sandwich ELISA system was developed to measure sCD22. The 96 well flat-bottomed plate (Nunc) was coated by the anti-CD22 mouse antibody, and 1 μg/well of RFB4 diluted by 100 μl of phosphate buffered saline (PBS) was incubated for 2 hours at room temperature. After washing the plate twice with 0.02% Tween PBS (T-PBS), the plate was blocked with 1% bovine serum albumin (BSA)-PBS to preclude nonspecific binding, then washed twice with T-PBS. The samples were then diluted with PBS, and the standard proteins diluted with 10% fetal bovine serum (FBS). PBS was then added and incubated 15˜20 hours. After washing with T-PBS three times, 50 ng/well of the second anti-CD22 antibody, SHCL1 (BD Pharmingen) biotinylated with Sulfosuccinimidyl1-6-(biotinamide) Hexanonate (Pierce) was added to the plate. In some examples, HIB22 (Sigma-Aldrich Corp., St. Louis, Mo.) was used instead of SHCL1. After three more washes with T-PBS, 100 μl of 10,000 fold diluted Avidine-HRP solution (Biosource) was added and incubated 1 hour at room temperature. After three more washes with T-PBS, 100 μl of TMB solution (Pierce) and 100 μl of H₂O₂ were added and incubated for 5 minutes, followed by the addition of 100 μl of 2N H₂SO₄ to stop the color development. The levels of sCD22 were determined by measuring the OD value at 450 nm.

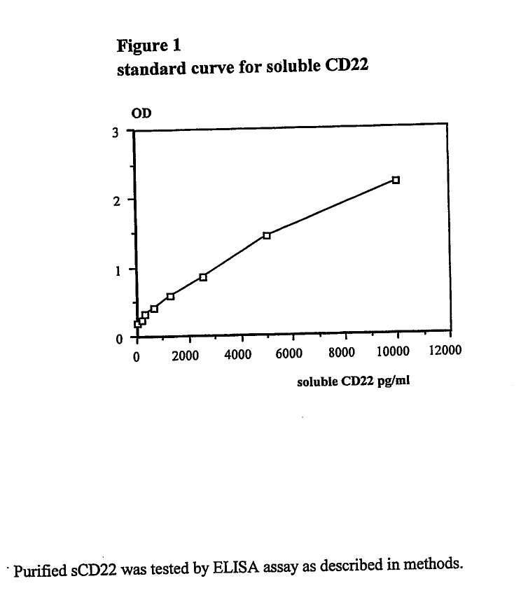

FIG. 1 shows the typical standard curve obtained using this system Soluble CD22 was measurable from 0.019 to 10 ng/ml. The inter-plate coefficient of variance (CV) at 0.1 ng/ml and 1 ng/ml were both within 10%, and the intra-plate CV at 0.1 ng/ml and 1 ng/ml were also within 10%. The levels of sCD22 were inhibited to around 50% by 100 ng/ml of BL22, which was the possible maximum concentration in the subjects' samples.

Levels of sCD22 in Subjects with HCL and CLL Prior to Treatment

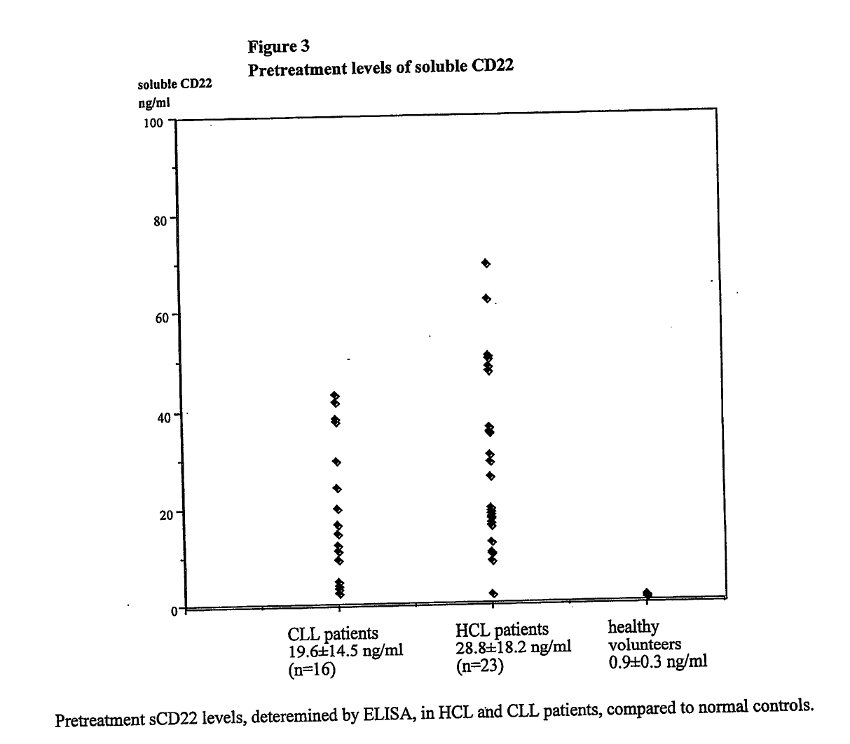

FIG. 3 shows the pre-treatment levels of plasma sCD22 in subjects with HCL and CLL, as well as in healthy volunteers who had not been diagnosed with HCL or CLL. The pre-treatment plasma levels of sCD22 in subjects averaged at 28.76±18.21 ng/ml, ranging from 1.72 to 69.62 ng/ml for HCL subjects (n=23), and averaged at 19.62±14.45 ng/ml, ranging from 2.41 to 43.32 ng/ml for CLL subjects (n=16). The plasma levels of sCD22 in healthy volunteers averaged at 0.90±0.28 ng/ml, ranging from 0.5 to 1.22 ng/ml (n=9). Thus, the plasma levels of sCD22 in subjects with HCL or CLL were significantly higher than those of healthy volunteers (both, p<0.001). Hence, in this example, when the “normal” range of sCD22 was 0.5-1.22 ng/ml, all subjects with HCL or CLL had a sCD22 level above the normal level, and in many instances substantially above the normal range. For example, a result associated with HCL and CLL can be any result above the normal range, or a sCD22 level of at least 1.5, 2, 3, 5, or even 10 times the top value of the normal range. Different cut off values can be selected depending on the desired sensitivity and specificity of the test.

Time Course of sCD22 Levels in Subjects Receiving BL22 Treatment

Soluble CD22 levels were used to monitor tumor burden of subjects with CLL and HCL who received immunotoxin BL22 therapy. Cycles of BL22 were from 3 weeks to several months apart, and ranged in dose and level from 10 to 50 μg/Kg four times per day for three days per cycle. Of the 21 subjects with HCL who received BL22, 16 subjects were evaluable, 11 subjects had a complete remission, two subjects had a partial remission, and three subjects received low dose of BL22 or had preexisting neutralizing antibody.

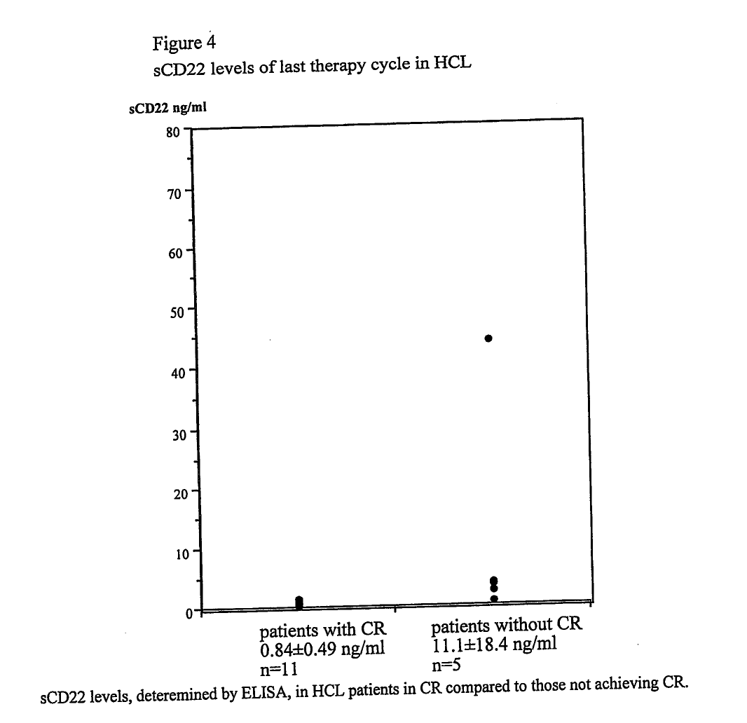

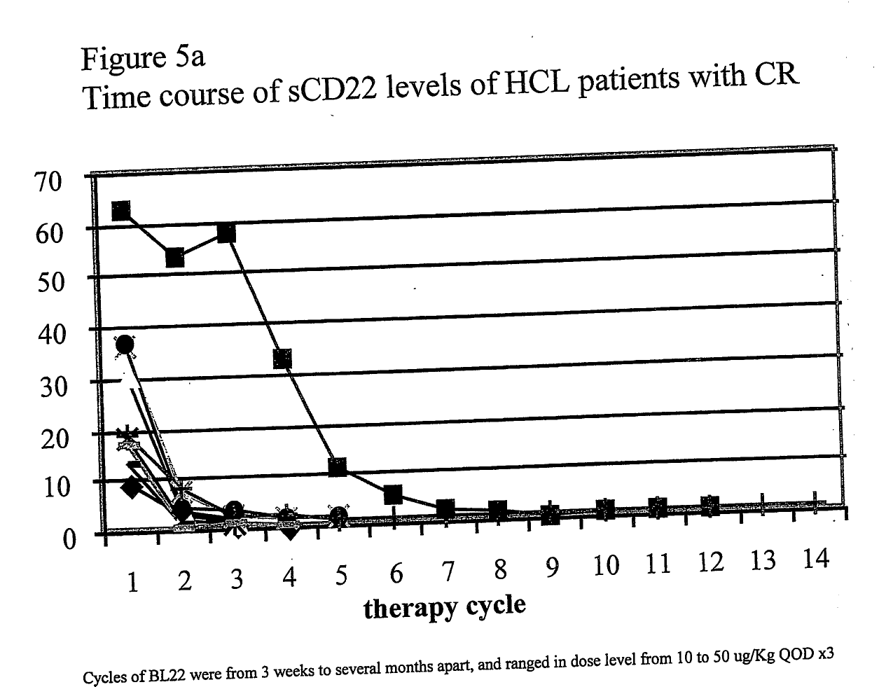

FIG. 4 shows the sCD22 levels of subjects just before their last cycle of therapy. Subjects with complete remission showed a low level of sCD22 that was similar to the level of healthy volunteers. In contrast, subjects without complete remission had higher levels of sCD22 than healthy volunteers and subjects with complete remission.

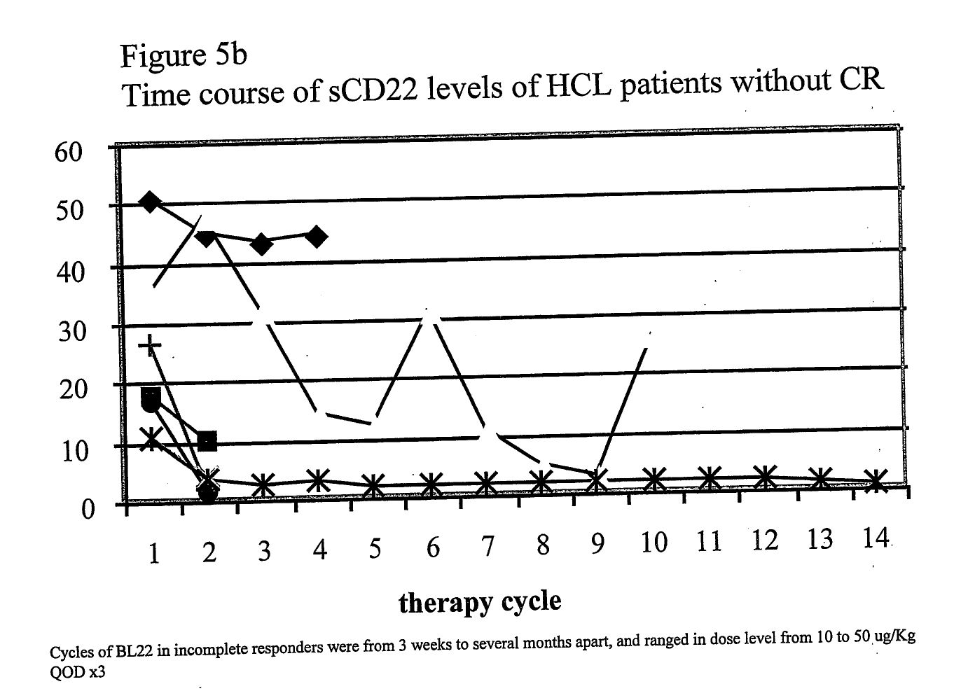

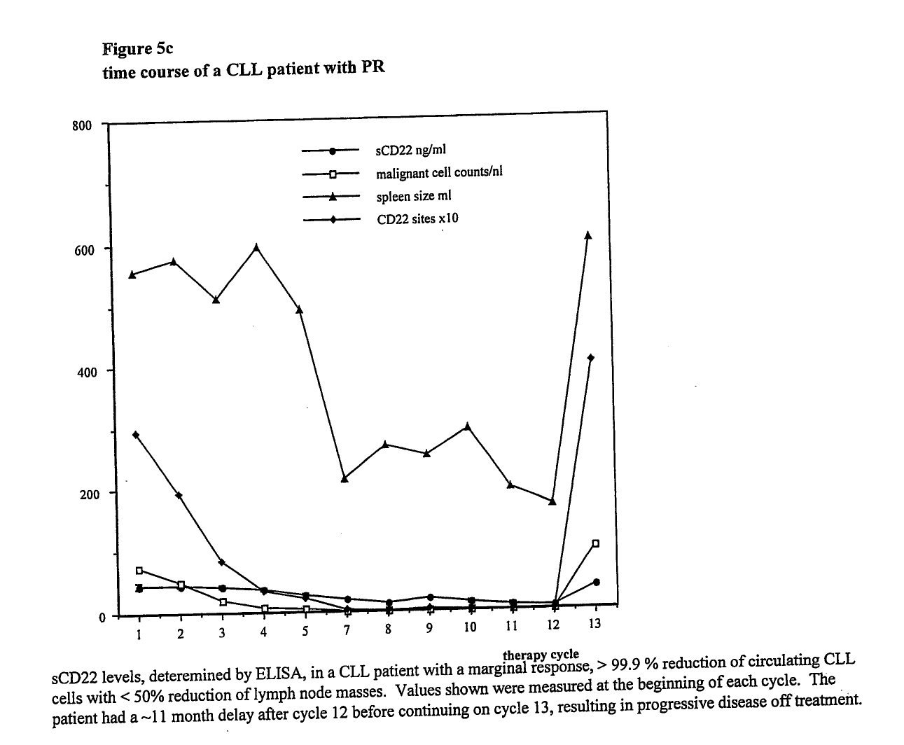

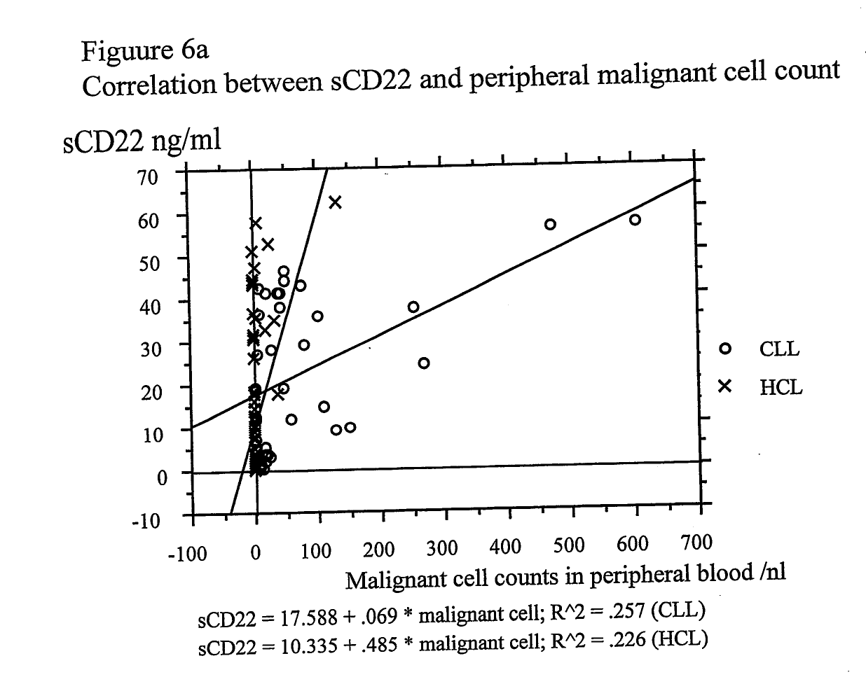

FIG. 5a shows a time course of the sCD22 levels of a subject with HCL who showed complete remission after a second course of therapy. The soluble CD22 levels decreased dramatically after initial therapy, and remained at a lower level throughout the time course. FIG. 5b shows the time course of a subject who showed a partial remission. The sCD22 level decreased more slowly compared to the former subject (complete remission) and fluctuated during the BL22 therapy. Before the 9th therapy cycle, the subject had relapsed disease. This was confirmed by flow-cytometry analysis; the level of sCD22 increased compared to the former level (during partial remission).

Further, in another two relapsed HCL subjects, sCD22 increased consistent with relapsed tumor burden. FIG. 5c shows the time course of a CLL subject with a partial remission. This subject exhibited >99.9% reduction of circulating CLL cells with a <50% reduction of lymph node masses. sCD22 levels decreased consistent with malignant cells counts or spleen size after therapy. The therapy was stopped after the 8th cycle of therapy with a partial remission. After a three-month interval, the subject's disease relapsed, and sCD22 levels increased. The additional four therapy cycles were administered, resulting in another partial remission. One year later, the subject's disease relapsed again, and the subject showed an elevation of sCD22. The disease course suggested that the mechanism of relapse was not the clonal proliferation of malignant cells with lower numbers of surface CD22 antigens. In other words, when patients relapsed, their malignant cells still displayed the same level of cell surface CD22 as they did when first treated. Thus, the treatment did not select for a CD22-negative malignant cell population.

The mean value of sCD22 levels of diseased subjects prior to treatment with BL22 was 20 to 30 times higher than the mean value of healthy volunteers. The levels of sCD22 were elevated in two subjects with variant HCL, which were negative for CD25. After treatment, the levels of sCD22 decreased dramatically in subjects who had complete remission or partial remission. In contrast, the levels of sCD22 did not decreased in the subjects who did not exhibit complete remission or partial remission.

There was one exception, a subject with CLL who exhibited a decrease of sCD22 level, while the leukemic cell count did not decrease. In this case, the immunotoxin-targeting cell surface CD22 antigen could kill only the malignant cells having high numbers of CD22 antigens and the cells having lower numbers of CD22 increased as the resistant cells against immunotoxin.

Generally, sCD22 levels were well correlated with the factors more traditionally representative of tumor burden, such as leukemic cell count, spleen size, and total count of surface CD22 antigen. Thus, soluble CD22 appears to be an excellent marker of tumor burden.

Static Analysis of the Correlation of sCD22 Levels with Tumor Burden

The difference of sCD22 levels between subjects with CLL or HCL and healthy volunteers was analyzed statically using a t-test. The correlation of sCD22 levels to total lymphocyte counts in peripheral blood, malignant cell counts calculated by the results of flow-cytometry, and spleen size was analyzed statically for each subject The correlation of sCD22 to the calculated total number of surface CD22 antigens was also analyzed.

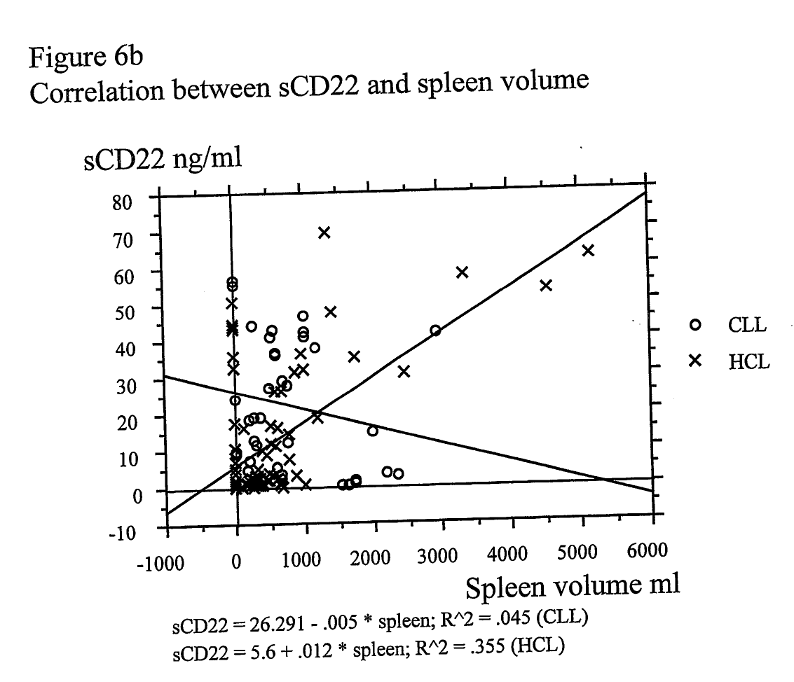

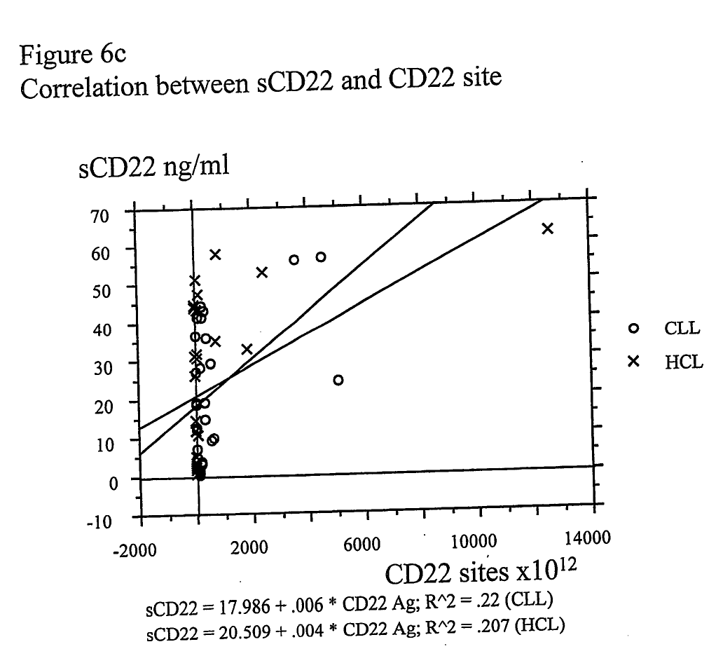

The levels of sCD22 of all of the subjects correlated well to malignant cell counts in peripheral blood and calculated spleen sizes (FIGS. 6a and 6b). Better correlations were seen between sCD22 levels and spleen sizes in HCL subjects, and between sCD22 levels and total malignant cell counts in CLL subjects. The main lesions of diseases are spleen in HCL subjects and peripheral blood in CLL subjects; these different disease characteristics may cause the differences in correlation.

FIG. 6c shows the correlation between sCD22 levels and total counts of surface CD22 antigen. HCL and CLL subjects can be recognized easily on the graph, which indicates that sCD22 was more easily produced from peripheral blood leukemic cells than malignant cells in spleen. Patients with large malignant cell counts in the blood had higher sCD22 levels than patients with low malignant counts in the blood and large spleens.

Western Blot Analysis to Determine the Size of sCD22

To detect the sCD22 in subjects' serum by Western blot, the sCD22 protein was collected on a cyanogen bromide—(CNBR) activated sepharose (Bio Rad) column loaded with RFB4. Briefly, the cyanogen bromide-activated sepharose is reacted with a protein of interest, which then becomes covalently attached to the resin. If this protein is RFB4 (an antibody specific for CD22), the resulting column can be used to purify sCD22 from serum or other samples.

A 2 mg sample of RFB4 was bound to 100 μl of CNBR-activated sepharose in 10 ml of 1 M sodium borate pH 8.0 by a recirculation manner. 500 ml of culture supernatant (10% FBS-RPMI1640) of Hairy cell leukemia cell line, Eskol (provided by Dr. M. Taylor, Indiana University, Ind.), and a diluted serum sample from a subject with HCL, were loaded to 25 μl of RFB4 attached sepharose columns. After being washed by PBS, the captured protein was eluted using sample buffer containing 5% of 2-mercaptoethanol. The samples were electrophoresed on a SDS-PAGE gel, then transferred to a nylon membrane (Immobilon-P, Millipore). After blocking with 0.5% of BSA, TBST solution, the membrane was incubated in 3 μg/ml of RFB4 antiserum for one hour. sCD22-RFB4 complex was visualized using a HRP-labeled anti-mouse IgG and IgA polyclonal secondary antibody (Biosource) followed by chemoilluminescence detection (ECL, NEN).

After purifying sCD22 protein from supernatant of Eskol cell line and subjects serum, Western blot analysis showed the major bands of sCD22 protein around 100 kDa. The purified product from FBS or serum of a healthy volunteer did not have the sCD22 bands. Thus, without being bound by theory, since the membrane-bound CD22 is approximately 135 kDa, sCD22 likely represents a truncated form of CD22 that lacks the transmembrane domain.

EPOCH Treatment

Together with the previous data, this experiment shows that sCD22 is a useful marker in following CLL, HCL and non-Hodgkin's lymphoma, and other B-cell malignancies, as well.

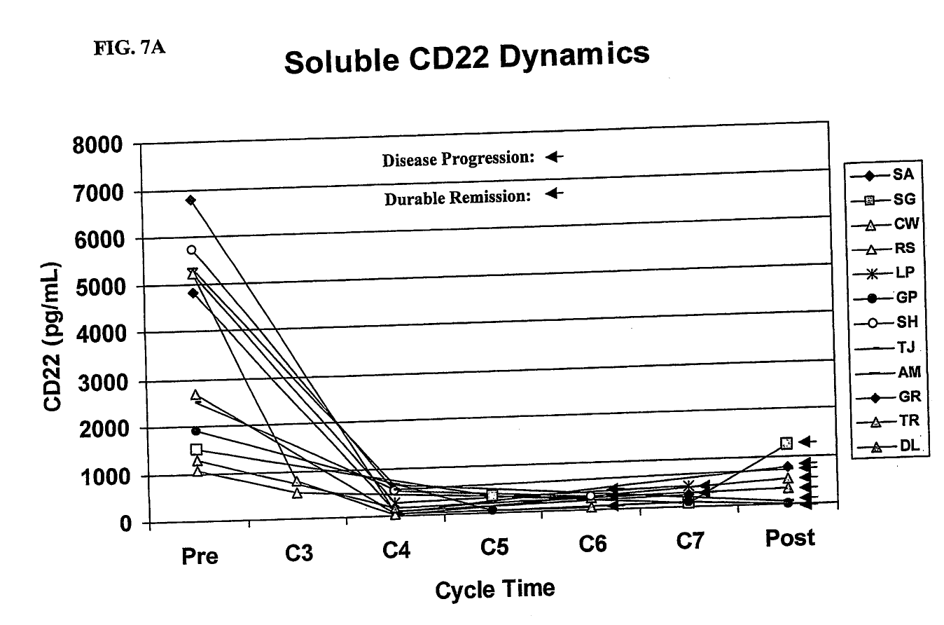

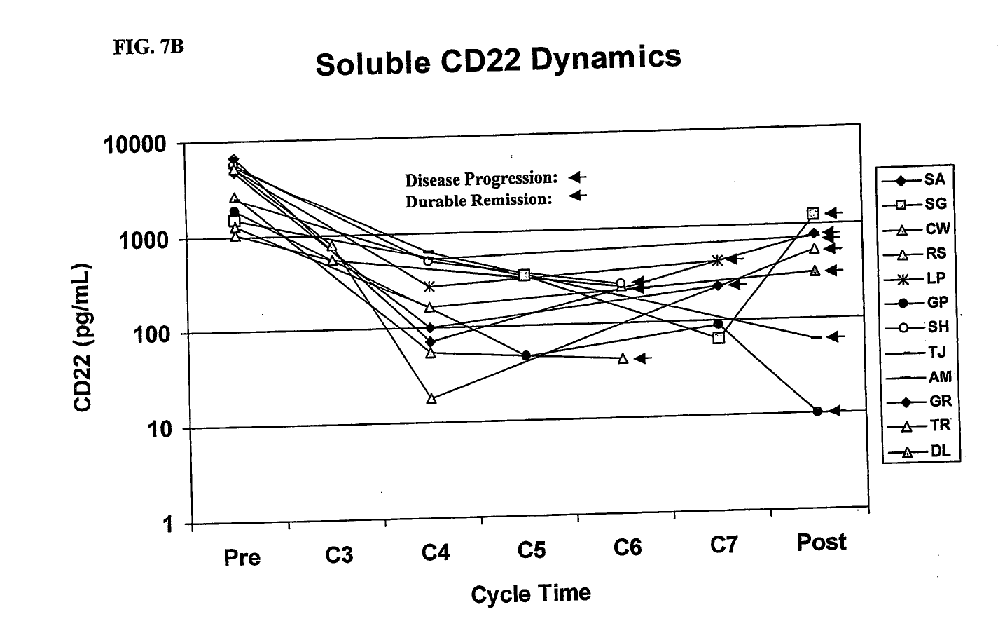

Twelve subjects received EPOCH treatment. All subjects had previously been untreated for large B-cell lymphomas. Six subjects each were HIV negative and positive. Subject demographics included a median (range) age of 43 (25-64) and advanced stage in 58%. Serum was analyzed for soluble CD22 levels at the following time points: pre-treatment, during treatment at cycles 3, 4, 5, 6 and 7, and post-treatment. In FIGS. 7A and 7B, the black arrows show six subjects with disease progression and the red arrows show six subjects in durable remissions. All subjects showed a decrement in soluble CD22 levels with treatment, and all subjects who relapsed showed an increase in sCD22 levels. However, optimal time points for relapsed subjects were not always available.

All subjects received dose-adjusted EPOCH as shown below in Table 2.

TABLE 2
EPOCH Starting Dose Level (Level 1)

| Drug | Dose | | Route | Treatment Days |
|---|---|---|---|---|
| Infused Agents | | | | |
| Etoposide | 50 | mg/m²/day | CIV | 1, 2, 3, 4 (96 hours) |
| Doxorubicin | 10 | mg/m²/day | CIV | 1, 2, 3, 4 (96 hours) |
| Vincristine² | 0.4 | mg/m²/day | CIV | 1, 2, 3, 4 (96 hours) |
| Bolus Agents | | | | |
| Cyclophosphamide | 750 | mg/m²/day | IV | 5 |
| Prednisone | 60 | mg/m²/bid | PO | 1, 2, 3, 4, 5 |
| G-CSF ® | 5 | μg/kg/day | SC | 6→ANC > 5000/μl |
| | | | | Past nadir |
| Next Cycle | | | | Day 21 |

Thus, sCD22 assessment correlated not only with patient response to treatment, but also with early relapse.

Although the foregoing examples describe uses for sCD22 in detecting, diagnosing, assessing, monitoring, and prognosing CLL and HCL, other B-cell lymphomas and B-cell leukemias are also associated with increased expression of cell-surface CD22, and can be identified, diagnosed, monitored, and prognosed using the methods described herein. For example, non-HCL, non-CLL B-cell lymphomas and B-cell leukemias can be detecting, diagnosing, assessing, monitoring, and prognosing by assessing soluble CD22.

This disclosure provides methods of using previously unknown soluble forms of CD22 (sCD22) present in the serum of subjects with B-cell leukemias and lymphomas to assess tumor burden in the subjects. The disclosure further provides methods of diagnosing or prognosing development or progression of a B-cell lymphoma or leukemia in a subject, including detecting sCD22 in a body fluid sample taken or derived from the subject, for instance serum. It will be apparent that the precise details of the methods described may be varied or modified without departing from the spirit of the described disclosure. We claim all such modifications and variations that fall within the scope and spirit of the claims below.

1. An assay for assessing B-cell leukemia or B-cell lymphoma in a subject, comprising:
measuring a level of soluble CD22 in a biological sample from the subject; and
correlating the level of soluble CD22 with a presence or activity of the B-cell leukemia or B-cell lymphoma.

2. The assay of claim 1, wherein the assay comprises diagnosing the B-cell leukemia or B-cell lymphoma by determining whether the soluble CD22 level is elevated above a predetermined level associated with the presence of B-cell leukemia or B-cell lymphoma.

3. The assay of claim 1, wherein the assay comprises determining tumor burden of the B-cell leukemia or B-cell lymphoma by detecting a change in the level of soluble CD22, wherein an increase in the level of soluble CD22 indicates an increase in a tumor burden of the B-cell leukemia or B-cell lymphoma and a decrease in the level of the soluble CD22 indicates a decrease in the tumor burden of the B-cell leukemia or B-cell lymphoma.

4. The assay of claim 1, wherein the assay comprises an assay for predicting development of the B-cell leukemia or B-cell lymphoma in the subject, wherein an elevation in the level of the soluble CD22 above a predetermined level indicates an increased likelihood of the subject developing B-cell leukemia or B-cell lymphoma.

5. The assay of claim 1, wherein measuring a level of soluble CD22 comprises making multiple measurements of the level of soluble CD22; and wherein a change in the level of the soluble CD22 is an indication of changing tumor burden in the subject.

6. The assay of claim 1, wherein the B-cell leukemia or B-cell lymphoma is hairy cell leukemia or chronic lymphocytic leukemia.

7. The assay of claim 6, wherein the B-cell leukemia is hairy cell leukemia.

8. The assay of claim 6, wherein the B-cell lymphoma is chronic lymphocytic leukemia.

9. The assay of claim 2, wherein the predetermined value is a normal value determined in a population of subjects who do not have a known B-cell leukemia or B-cell lymphoma.

10. The assay of claim 1, wherein the biological sample is a serum sample.

11. The assay of claim 1, wherein measuring a level of soluble CD22 in a biological sample comprises contacting the biological sample with a specific binding agent to form a detectable complex, and quantitating the detectable complex.

12. The assay of claim 11, wherein the specific binding agent is detectably labeled.

13. The assay of claim 1, wherein the assay comprises an assay for determining an appropriate anti-tumor therapy for the subject, wherein the anti-tumor therapy is administered to the subject, and the anti-tumor therapy is continued if the level of soluble CD22 subsequently declines.

14. The assay of claim 1, wherein the assay comprises an assay for identifying an anti-tumor agent for treatment of B-cell leukemia or B-cell lymphoma, and the assay further comprises administering a test agent to the subject and subsequently determining whether the level of soluble CD22 declines, wherein a decline in the level of soluble CD22 indicates the test agent is an anti-tumor agent.

15. The assay of claim 1, wherein the assay comprises an assay for measuring clinical progression of B-cell leukemia or B-cell lymphoma in a subject known to have B-cell leukemia or B-cell lymphoma, wherein an increase in the level of soluble CD22 indicates an increase in tumor burden of the B-cell leukemia or B-cell lymphoma.

16. The assay of claim 1, wherein the assay comprises an assay for measuring clinical regression of B-cell leukemia or B-cell lymphoma in a subject known to have B-cell leukemia or B-cell lymphoma, wherein a decrease in the level of soluble CD22 indicates a decrease in tumor burden of the B-cell leukemia or B-cell lymphoma.

17. The assay of claim 1, wherein the assay comprises an assay for determining whether to initiate anti-tumor therapy in a subject who does not have other clinical evidence of B-cell leukemia or B-cell lymphoma, wherein the anti-tumor therapy is administered to the subject if the level of soluble CD22 is above a predetermined value associated with a predisposition to develop B-cell leukemia or B-cell lymphoma.

18. A kit for measuring a soluble CD22 level, comprising a specific binding agent that selectively binds to sCD22, and instructions for carrying out the method of claim 1.

19. The kit of claim 18, wherein the specific binding agent is an antibody or antibody fragment that selectively binds sCD22.

20. A method of testing a compound to determine whether it is useful in treating, reducing, or preventing B-cell lymphomas or B-cell leukemias or development or progression of B-cell lymphomas or B-cell leukemias, comprising:
determining if administration of a test compound lowers soluble CD22 levels in a subject; and
selecting a compound that so lowers soluble CD22 levels.

21. A method of monitoring progress of treatment for a B-cell lymphoma or B-cell leukemia in a subject comprising:
administering an anti-tumor compound or putative anti-tumor compound to the subject; and
monitoring a soluble CD22 level in the subject to determine whether the soluble CD22 level falls as an indication that the compound is reducing tumor burden in the subject.

22. A method for treating a B-cell lymphoma or B-cell leukemia in a subject, comprising:
obtaining a body fluid sample from a subject;
identifying an elevated level of soluble CD22 relative to a control; and
administering a therapeutically effective amount of an anti-cancer agent, thereby treating the subject or inhibiting the B-cell lymphoma or leukemia.

23. A method of diagnosing or prognosing development or progression of a B-cell lymphoma or B-cell leukemia in a subject, comprising:
contacting a body fluid sample from the subject with a CD22-specific binding agent;
detecting whether the binding agent is bound by the sample, thereby measuring the levels of the soluble CD22 present in the sample; and
comparing in the level of sCD22 in the sample to a control value, wherein the control value represents the level of soluble CD22 found an analogous sample from a subject not having a B-cell lymphoma or B-cell leukemia, or a standard soluble CD22 level in analogous samples from a subject not having a B-cell lymphoma or B-cell leukemia or not having a predisposition for developing a B-cell lymphoma or B-cell leukemia, wherein a sCD22 level greater than the control level is diagnostic or prognostic for development or progression of a B-cell lymphoma or B-cell leukemia in the subject.
